(19) United States
(12) Patent Application Publication
(10) Pub. No.: US 2005/0026296 A1
(43) Pub. Date: Feb. 3, 2005

(54) Diagnostic method based on lipid measuring parameter modulations/effector quotient profiles

(76) Inventors: Hanns-Wolf Baenkler, Herzogenaurach (DE); Dirk Schafer, Forchheim (DE)

Correspondence Address: GRIFFIN & SZIPL, PC, SUITE PH-1, 2300 NINTH STREET, SOUTH, ARLINGTON, VA, 22204, US

(21) Appl. No.: 10/727,568

(22) Filed: Dec. 5, 2003

(30) Foreign Application Priority Data
Jun. 5, 2002 (WO) PCT/EP02/06167
Jun. 5, 2001 (EP) 01113712.2

(51) Int. Cl. G01N 33/92

(52) U.S. Cl. 436/71

(57) ABSTRACT

The invention relates to a method for the diagnosing or for the confirmation or for the exclusion of constellations of risk factors, pathological states or predispositions thereto, and to a method for monitoring the course of therapies and for finding active substances for the treatment of pathological states and for finding substances which may cause such a pathological state, on the basis of lipid measurement parameter modulation/effector quotient profiles. Further, the invention relates to a measuring instrument for carrying out the above methods.

### CROSS-REFERENCE TO RELATED APPLICATIONS

This is a continuation of PCT/EP02/06167 filed Jun. 5, 2002, which claims priority on European Patent Application No. 01113712.2 filed Jun. 5, 2001. The entire disclosures of the above patent applications are hereby incorporated by reference.

### BACKGROUND OF THE INVENTION

1. Field of the Invention

The invention relates to a method for the confirmation or exclusion of constellations of risk factors, pathological states or predispositions thereto, and to a method for monitoring the course of therapies and for finding active substances for the treatment of pathological states and for finding active substances that can induce such a pathological state, based on lipid measurement parameter modulation/effector quotient profiles. Furthermore, the present invention relates to a measuring instrument for carrying out said methods.

2. Background Art

The presence of lipids/eicosanoids in the human body and their significance for potentially many human body functions was already recognized more than 100 years ago. Their biosynthesis and their affection by complex as well as by pure chemical compounds is already known since the Middle Ages (e.g. the pain-relieving effect of the crack willow extract) has been elucidated biochemically in increasing detail since the middle of the 20^{th} century (1-15). The presence of the lipids/eicosanoids and their impact on animals (also on amphibians, insects and microorganisms) and plants is also known (16-19). Furthermore, the knowledge of the different lipid/eicosanoid receptors and possible subtypes is growing increasingly. Also, the genes and their chromosomal localisations for some enzymes of the eicosanoid metabolism and the eicosanoid receptors are known (20-26).

To date, the detection of lipids/eicosanoids is achieved with different physical-chemical and immunological techniques (e.g. by gas chromatography, high-pressure liquid chromatography (HPLC), thin layer chromatography, radioimmunoassays (RIA), enzyme immunoassays (EIA)). These methods differ from each other with respect to their detection sensitivity and resolution capacity for different lipids/eicosanoids during measurement and to the maximal sample volume throughput (27).

The detection or the determination of enzymes involved in the synthesis or metabolism of lipids/eicosanoids is commonly achieved after gel electrophoretic separation of the cellular proteins by subsequent labelling using specific antibodies or on intact cells with radioactive or otherwise labelled lipids/eicosanoids or receptor ligands (agonists/antagonists). An immunocytological detection of the enzymes or receptors by immunocytological methods using suitable monoclonal or polyclonal antibodies is also possible. The detection or determination of enzymatic activity is achieved directly by measuring the degradation of suitable enzyme substrates, or indirectly by detecting secondary metabolites formed (20, 21, 24, 37, 29-30).

The detection or determination of mRNA for the enzymes and receptors that are involved in the synthesis or metabolism of lipids/eicosanoids or in their binding, can e.g. be achieved by RT-PCR (reverse transcriptase polymerase chain reaction) or Northern blotting.

Although analytical methods for the total or selective determination of certain eicosanoids are known (21-30), the lipids/eicosanoids have not yet, at least broadly, been used for clinical/diagnostic purposes (31,32). Only for certain eicosanoids called peptide leukotrienes is a test system commercially available (“CAST-Elisa” of Bühlmann-Laboratories, see also U.S. Pat. No. 5,487,977) that serves for the detection of elevated peptide leukotriene levels after in vitro stimulation with allergens in combination with general cell-activating substances (e.g. complement factors or cytokines). The degree of sensitization of the examined (allergic) patients is derivable from the amount of released lipids/eicosanoids, i.e. peptide leukotrienes (32). Also, the modulation of the lipid/eicosanoid levels in different clinical scenarios has been mentioned (33-37).

In their work, the different authors point out the measurability of modified lipid/eicosanoid amounts in the samples examined by them, and report that many of the examined clinical scenarios are correlated with elevated lipid/eicosanoid levels. In general, however, nothing is mentioned with respect to a lipid/eicosanoid pattern or profile being modified versus a normal state. Moreover, only the actual state of lipids/eicosanoids is determined. However, e.g. the impact of a modulation (stimulation/inhibition) of the eicosanoid synthesis of the biological material in vitro is not taken into consideration for diagnostic purposes (with the exception of CAST-ELISA already mentioned above). Until now, enzymes and receptors of the lipid/eicosanoid metabolism have not been used for diagnostic purposes, neither in the actual state nor in the modulated state.

The problem addressed by the invention follows from this state of the art.

### SUMMARY OF THE INVENTION

The invention thus relates to a method and apparatus for the confirmation or exclusion of pathological states or predispositions based on lipid measurement parameter modulation/effector quotient profiles.

In a first preferred embodiment of the invention, the method comprises the steps of:
(a) providing a sample from an organism to be investigated and dividing the sample into a plurality of sufficient equal part-samples to allow for measurement of a plurality of values for each of a plurality of lipid measurement parameters A, B, C, . . . ;
(b) measuring a plurality of zero values A₀, B₀, C₀, . . . in the absence of a modulating effector; measuring a plurality of indicator values Aₘₐₓ, Bₘₐₓ, Cₘₐₓ, . . . in the presence of a modulating effector or an indicator substance; and measuring a plurality of values for a further modulation A₂, B₂, C₂, . . . in the presence of a further modulating effector;
(c) calculating a plurality of quotients of the measurements Aₘₐₓ/A₀, A₂/A₀; BₘₐₓB₀, B₂/B₀; Cₘₐₓ/C₀, C₂/C₀; . . . for each lipid measurement parameter A, B, C, . . . of the sample from the organism to be investigated; and dividing the quotients by the corresponding values of one or more standard group(s), resulting in standardized modulation quotients which in their totality form a standardized modulation quotient profile for the organism to be investigated;
(d) calculating a plurality of quotients A₀/B₀, B₀/A₀, A₀/C₀, C₀/A₀, B₀/C₀, C₀/B₀ . . . in any combination from the zero values A₀, B₀, C₀ . . . ; and a plurality of quotients Aₘₐₓ/Bₘₐₓ, Bₘₐₓ/Aₘₐₓ, Aₘₐₓ/Cₘₐₓ, Cₘₐₓ/Aₘₐₓ, Bₘₐₓ/Cₘₐₓ, Cₘₐₓ/Bₘₐₓ . . . in any combination from the indicator values Aₘₐₓ, Bₘₐₓ, Cₘₐₓ . . . ; and a plurality of quotients A₂/B₂, B₂/A₂, A₂/C₂, C₂/A₂, B₂/C₂, C₂/B₂ . . . in any combination from the values for further modulation A₂, B₂, C₂ . . . ; and then dividing the values obtained for the organism to be investigated by a plurality of corresponding values obtained for one or more standard group(s) to obtain a plurality of standardized effector quotients which in their totality form a standardized effector quotient profile for the organism to be investigated; and
(e) diagnosing, confirming, or excluding a constellation of risk factors, a pathological state, or a predisposition thereto by comparing the standardized modulation quotient profile and the standardized effector quotient profile of the organism to be investigated with that of a corresponding investigation group in which the constellation of risk factors of interest, the pathological state, or the predisposition is present.

In a second preferred embodiment of the invention, the lipid measurement parameters of the first embodiment are selected from the group consisting of measurement parameters for unsaturated fatty acids, degrading enzymes and synthesizing enzymes for unsaturated fatty acids, nucleic acids coding for degrading enzymes and synthesizing enzymes for unsaturated fatty acids, receptors for unsaturated fatty acids, and nucleic acids coding for receptors for unsaturated fatty acids.

In a third preferred embodiment of the invention, the unsaturated fatty acids of the second embodiment are selected from the group consisting of platelet-activating factor and eicosanoids.

In a fourth preferred embodiment of the invention, the eicosanoids of the third embodiment are selected from the group consisting of peptide leukotrienes, prostaglandin E2, thromboxane A2 and thromboxane B2.

In fifth preferred embodiment of the invention, the modulating effector or indicating substance of the first embodiment is selected from the group consisting of arachidonic acid, chemotactic peptides, anti-IgE, lipopolysaccharide, and interleukin.

In a sixth preferred embodiment of the invention, the modulating effector of the first embodiment is a substance which may cause a pathological state or is involved in the onset or development thereof.

In a seventh preferred embodiment of the invention, the pathological state of the sixth embodiment is selected from tumours, cystic fibrosis, polyposis, bronchial asthma, an intolerance, coagulation defects, overcoming of infection, and an inflammation.

In an eighth preferred embodiment of the invention, the pathological state of the sixth embodiment is inflammatory and neoplastic change of the gastrointestinal tract.

In a ninth preferred embodiment of the invention, the intolerance of the sixth embodiment is a food, food additive or drug intolerance or an allergy, or wherein said coagulation defects represent the basis for thromboses or haemorrhages or thrombophilia, or wherein said overcoming of infection is a resistance to bacterial or viral or mycotic elements, e.g. associated with bacterial, viral or mycotic mucositis, or wherein the inflammation is encephalitis, sinusitis, rhinitis, neurodermatitis, Crohn's disease or ulcerative colitis.

In a tenth preferred embodiment of the invention, the drug intolerance of the ninth embodiment is an analgesic intolerance or said allergy is a pollen, spore, mite, wasp or bee venom allergy.

In an eleventh preferred embodiment of the invention, the analgesic intolerance of the tenth embodiment is intolerance of acetylsalicylic acid.

In a twelfth preferred embodiment of the invention, one or more, optionally labeled eicosanoid(s) or the dye 9-diethylamino-5H-[alpha]phenoxazin-5-one is/are used to determine the lipid measurement parameters.

In a thirteenth preferred embodiment of the invention, immobilized probes are used to determine the lipid measurement parameters, and the immobilized probes are selected from the group consisting of antibodies or functional fragments thereof against degrading enzymes or synthesizing enzymes of unsaturated fatty acids or against receptors for unsaturated fatty acids, and nucleic acids which hybridize onto nucleic acids which code for degrading enzymes or synthesizing enzymes of unsaturated fatty acids or for receptors for unsaturated fatty acids.

In an fourteenth preferred embodiment of the invention, the antibodies of the thirteenth embodiment are selected from the group consisting of polyclonal, monoclonal and single-chain antibodies, and said nucleic acids are selected from cDNA, mRNA and oligonucleotides.

In an fifteenth preferred embodiment of the invention, the immobilized probes of the thirteenth embodiment form an addressable pattern on a surface.

A sixteenth preferred embodiment of the invention is a method for monitoring the course of therapies of pathological states based on lipid measurement parameter modulation/effector quotient profiles, in which a method according to the first embodiment is carried out after the administration or in the presence of a suitable medicament.

A seventeenth preferred embodiment of the invention is a method for finding active substances for the treatment of pathological states based on lipid measurement parameter modulation or effector quotient profiles, in which a method according to the first embodiment is carried out after the administration or in the presence of a candidate active substance.

An eighteenth preferred embodiment of the invention is a method for finding substances able to induce a pathological state based on lipid measurement parameter modulation or effector quotient profiles, in which a method according to the first embodiment is carried out after an administration/application or in the presence of such a substance.

A nineteenth preferred embodiment of the invention is the method of the first embodiment, wherein said sample contains leukocytes.

A twentieth preferred embodiment of the invention is the method of the first embodiment, wherein said lipid measurement parameters are selected from the group consisting of measurement parameters for ceramide; ceramide-1-phosphate; sphingosine; sphingosine-1-phosphate; phosphatidic acid; diacylglycerol; lysophosphatidic acid; the phosphatidylinositol phosphates; and enzymes modifying ceramide, ceramide-1-phosphate, sphingosine, sphingosine-1-phosphate, phosphatidic acid, diacylglycerol, lysophosphatidic acid, or the phosphatidylinositol phosphates.

A twenty-fist preferred embodiment of the invention is an apparatus for obtaining lipid measurement parameter modulation or effector quotient profiles, comprising:
(a) means for providing a sample from an organism;
(b) means for measuring a plurality of values for each of a plurality of lipid measurement parameters A, B, C, . . . , the means for measuring comprising:
   i. means for measuring a plurality of zero values A₀, B₀, C₀, . . . in the absence of a modulating effector;
   ii. means for measuring a plurality of indicator values Aₘₐₓ, Bₘₐₓ, Cₘₐₓ, . . . , in the presence of a modulating effector or an indicator substance; and
   iii. means for measuring a plurality of values for a further modulation A₂, B₂, C₂, . . . , in the presence of a further modulating effector;
(c) means for calculating, the calculating means comprising:
   i. means for obtaining a standardized modulation quotient profile, comprising a plurality of standardized modulation quotients, by dividing plurality of quotients of the measurements Aₘₐₓ/A₀, A₂/A₀; BₘₐₓB₀, B₂/B₀; Cₘₐₓ/C₀, C₂/C₀; . . . for each lipid measurement parameter A, B, C, . . . of the sample from the organism to be investigated and dividing the quotients by the corresponding values of one or more standard group(s); and
   ii. means for obtaining a standardized effector quotient profile, comprising a plurality of standardized effector quotients, by calculating a plurality of quotients A₀/B₀, B₀/A₀, A₀/C₀, C₀/A₀, B₀/C₀, C₀/B₀ . . . in any combination from the zero values A₀, B₀, C₀ . . . ; and a plurality of quotients Aₘₐₓ/Bₘₐₓ, Bₘₐₓ/Aₘₐₓ, Aₘₐₓ/Cₘₐₓ, Cₘₐₓ/Aₘₐₓ, Bₘₐₓ/Cₘₐₓ, Cₘₐₓ/Bₘₐₓ . . . in any combination from the indicator values Aₘₐₓ, Bₘₐₓ, Cₘₐₓ . . . , and a plurality of quotients A₂/B₂, B₂/A₂, A₂/C₂, C₂/A₂, B₂/C₂, C₂/B₂ . . . in any combination from the values for further modulation A₂, B₂, C₂ . . . ; and then dividing the values obtained for the organism to be investigated by a plurality of corresponding values obtained for one or more standard group(s); and
(d) means for comparing the standardized modulation quotient profile and the standardized effector quotient profile of the organism to be investigated with that of a corresponding investigation group.

A twenty-second preferred embodiment of the invention is the apparatus of the twenty-first embodiment, wherein one or more of the measuring means comprises a surface on which probes defined for determination of the lipid measurement parameters are immobilized, which probes are selected from the group consisting of antibodies or functional fragments thereof against degrading enzymes or synthesizing enzymes of unsaturated fatty acids, antibodies or functional fragments thereof against receptors for unsaturated fatty acids, and nucleic acids which hybridize onto nucleic acids which code for degrading enzymes or synthesizing enzymes of unsaturated fatty acids or for receptors for unsaturated fatty acids, wherein the antibodies are preferably selected from polyclonal, monoclonal and single-chain antibodies, and the nucleic acids are preferably selected from cDNA, mRNA and oligonucleotides.

A twenty-third preferred embodiment of the invention is the apparatus of the twenty-second embodiment, wherein the probes form an addressable pattern on the surface.

### DETAILED DESCRIPTION OF THE PREFERRED EMBODIMENTS

It is clear that the error propagation is considered when calculating the lipid measurement parameter modulation/effector quotients. Further, it can broadly be stated that a lipid measurement parameter modulation/effector quotient is striking if it is about 5-10% higher or lower than the corresponding standard value. As a further decision criterion for the differentiation between normal and striking, one may also use the two-fold standard deviation 2σ of the respective normal (standard) lipid measurement parameter modulation/effector quotient can serve. If the value concerned is modulated over the standard value ±2σ, it is considered to be striking.

It is clear that the term “standard group” or “investigation group” is relative. The “standard group” may also be formed by the single organism to be investigated itself or by parts thereof (e.g. tissues, body fluids), e.g. when healthy state measurements are used later for the standardization of measurements taken in an acute pathological state, or simply for determining whether any modifications have occurred. Analogously, this is also valid for the “investigation group”, e.g. when values of an acute pathological state are compared with the actual state after therapy. It is also clear that the measurements for the standard groups and for the investigation groups or the measurements only for a single organism to be investigated in the normal state and in the state of acute illness, respectively, may also be saved in a database and used on demand for standardization or for comparison. If such measurements should not be available, they can easily be obtained by applying the method according to the invention to corresponding “normal” and “pathological” states or to corresponding “standard groups” and “investigation groups”.

Until now, the determination of lipid measurement parameters has almost exclusively been done for single tasks and mostly without any clinical/diagnostic allusion. If there is a diagnostic problem at all (e.g. with “CAST-ELISA” of Bühlmann-Laboratories), however, only one lipid measurement parameter (here: the peptide leukotrienes), or e.g. only the basic amounts of eicosanoid 1 and 2 and perhaps 3 or enzyme 1 and 2 etc. or the synthesis of eicosanoid 1 is determined (32). In contrast, the method according to the invention particularly facilitates to determine and to clinically/diagnostically evaluate the ratio of different lipid measurement parameters to each other (so-called balances), leading to a classification in risk constellations.

The lipid measurement parameter modulation/effector quotient profiles obtained by the method according to the invention enable the differentiation of clinical scenarios, the “monitoring” of therapies, the estimation of anti-inflamma-tory/pro-inflammatory effects of matter, substances and materials, and finally also the inflammatory risk assessment of known und unknown matter/substance groups in vitro. By using new technologies (Micro-Multi-Array-Methods), also very small sample amounts may now be analysed according to the method described and important conclusions may be drawn there from.

As already mentioned, different clinical scenarios may be diagnosed or characterized according to the invention on the basis of the respective lipid ratios being characteristic thereof.

For example, the course of a desensitization against allergens or of a desactivation to acetylsalicylic acid (aspirin) can be measured directly without disturbing the patient. Both, the modulation and the measurement are performed ex-vivo. An in vivo measurement is not necessary. Contrary to the state of the art, the method according to the invention is based on the acquirement of information by comparing at least two lipid measurement parameters in the modulated (stimulated/inhibited) and unmodulated state. A risk constellation cannot reliably be determined by using one lipid measurement parameter alone (with CAST-ELISA (32), e.g. only 1 lipid measurement parameter is determined), but only by using the lipid measurement parameter modulation/effector quotient profiles obtained by the method according to the invention which e.g. consider the ratio of e.g. prostaglandins to leukotrienes.

Further advantageous or preferred embodiments of the invention are subject-matter of the dependent claims.

According to one embodiment of the method according to the invention, the lipid measurement parameters in step (b) are selected from measurement parameters for cell-signaling lipids such as ceramide and sphingosine and their phosphates, phosphatidic acid, diacylglycerol, lysophosphatidic acid, and the phosphatidylinositol phosphates.

According to one embodiment of the method according to the invention, the lipid measurement parameters in step (b) are selected from measurement parameters for unsaturated fatty acids, degrading and synthesizing enzymes for unsaturated fatty acids and nucleic acids (mRNA) coding therefor, and from those for receptors for unsaturated fatty acids and nucleic acids coding therefor (mRNA).

For example, the unsaturated fatty acids are selected from platelet-activating factor (12) and eicosanoids, e.g. peptide leukotrienes (=pLTs, e.g. LTC4, LTD4, LTE4), prostaglandin E2 (PGE2), thromboxane A2 and thromboxane B2 (=TXB2).

According to a further embodiment of the method according to the invention, arachidonic acid or a chemotactic peptide such as e.g. fMLP is used as maximal modulating effector (indicator substance) in step (b) as defined above.

According to a further embodiment of the method according to the invention, a substance (e.g. viruses, bacteria or other organisms such as yeast, fungi or components thereof, chemicals in the broadest sense such as solvents or dyes, allergens in the broadest sense, in particular also from biological origin, pharmaceuticals, toxins, in particular also from biological origin) is used as a further modulating effector in step (b) as defined above which may cause a striking state, e.g. a pathological state, or is involved in the onset or development thereof.

The method according to the invention is e.g. suitable for the diagnosis or for the confirmation or exclusion of the following pathological states and for the estimation of a predisposition thereto: tumours, e.g. bronchial tumours, cystic fibrosis, polyposis, e.g. polyposis nasi et sinuum, bronchial asthma, intolerance of food, food additives or drugs, e.g. of analgesics such as acetylsalicylic acid (aspirin), allergies such as pollen, spore, mite, wasp und/or bee venom allergy, e.g. as allergic asthma, different inflammations such as encephalitis, sinusitis, rhinitis, neurodermatitis, Crohn's disease, ulcerative colitis, diarrhoeas, overcoming of infection, e.g. on mucosas, e.g. for resistance to bacterial, viral or mycotic elements, e.g. in bacterial, viral or mycotic mucositis, in susceptibility for infections, coagulation defects, e.g. thromboses, haemorrhages or thrombophilia.

Particularly preferred pathological states relate to the gastrointestinal tract. It is known for example, that the incidence of inflammatory intestinal diseases ranges from 5-10/100,000, with an indicated prevalence of 100/100,000. Interestingly, Caucasians are affected more often than blacks or Hispanics. The risk factors under discussion include food as well as food ingredients and additives that are taken with the food. Up to 70% of the granulocytes circulating in the blood pass the intestinal epithelium within 48 hours and may thus come into direct contact with food allergens.

In the field of food intolerances, particularly the following organs are affected: skin (ca. 40%), respiratory tract (ca. 23%), gastrointestinal tract (ca. 20%). Occasionally, these intolerances are accompanied by impairments of the cardiovascular system (ca. 10%), by mild shock conditions up to anaphylactic shock, as well as by migraine and by certain behaviour disorders such as the “hyperkinetic syndrome”. A significant part of the population suffers from food intolerances.

Further it is known that ulcerations of the mucosa of the gastrointestinal tract microscopically do not allow drawing conclusions about a special genesis. Also histological results in the absence of a definite detection of certain pathogens or in the absence of a detection of tumour markers do not allow making a clear diagnosis. In some cases, the basis may be inflammatory, toxic, or allergic reactions. However, the performing of conventional investigation methods such as e.g. prick testing, or the prescription of an allergic diet as primary diagnostics often only provides a low specificity, or sensitivity, or both. Here, the determination of an eicosanoid profile as suggested according to the invention leads to a clearly improved clarification of the pathological situation. For example, investigations with blood cells from patients with a food intolerance indicated that the actual causes could be identified from a substantial number of potential causative food articles which had been negatively prick tested before, by applying the method according to the invention and the in vitro provocation of the cells with these substances. The knowledge of these causative substances brought about by the invention led to a clear improvement of the clinical symptoms by omitting these substances in the course of a correspondingly ordered diet, which is an impressing confirmation of the successful establishment of a risk constellation according to the invention.

In the field of gastrointestinal ulcers, endoscopic investigations of patients taking non-steroidal anti-inflammatory drugs (NSAIDs) indicated a prevalence of 15-30%. Intestinal complications with clinical relevance were observed in 2%. The involvement of eicosanoids in these clinical scenarios has often been described in the literature. In particular, the intolerances of NSAIDs—possibly due to an imbalance of the eicosanoids—is very frequently spread and in view of 1000-2000 deaths/year in England/UK and of 2000-16000 cases in the US to be regarded as life-threatening. Thus, the potential of applying the method according to the invention to the investigation of biopsies and/or isolated blood cells is obvious. For example, own investigations indicated that patients with ulcers had lipid measurement parameters that in the sense of a risk constellation had to be regarded as being striking. Since an infiltration with inflammatory cells was observed in several inflammatory processes it seems a likely supposition that these inflammatory cells—also comprising cells of the blood—release the lipid mediators measured according to the invention and contribute to the damage of the intestinal mucosa.

Thus, it is particularly preferred according to the invention to apply the present method to the diagnosis or analysis of inflammatory and neoplastic diseases or changes of the gastrointestinal tract.

According to an embodiment of the method according to the invention, one or more, optionally labelled eicosanoid(s) or the dye 9-diethylamino-5H-benzo[alpha]phenoxazin-5-one (38-40) is/are used to determine the lipid measurement parameter.

According to an embodiment of the method according to the invention, immobilized probes selected from antibodies or functional fragments thereof against degrading or synthesizing enzymes of unsaturated fatty acids or against receptors for unsaturated fatty acids, nucleic acids which hybridize onto nucleic acids which code for degrading or synthesizing enzymes of unsaturated fatty acids or for receptors for unsaturated fatty acids, are used for the determination of the lipid measurement parameters.

In general, a probe is a recognizing molecule or a receptor that can specifically recognize und bind a ligand.

It is to be understood, that a “functional” fragment of an antibody is an antibody fragment which is able to bind to an antigen, but which does not need to be immunogenic, too.

Suitable antibodies are selected from polyclonal, monoclonal and single-chain antibodies.

Suitable nucleic acids are selected from cDNA, mRNA and oligonucleotides.

Preferably, the immobilized probes form an addressable pattern on a surface, resulting in a so-called biochip.

The invention further relates to a method for monitoring the course of therapies of striking states (e.g. pathological states) based on lipid measurement parameter modulation/effector quotient profiles, in which the method according to the invention as defined above is carried out after the administration/application (i.e. the medicament to be investigated is administered to the volunteers or the organism before sample collection) or in the presence (i.e. the medicament to be investigated is added to the sample after its withdrawal from a volunteer or organism) of a suitable medicament. This medicament may be regarded as being one of the “further modulating effectors” according to the invention. Of course, combinations of medicaments may also be used.

The invention further relates to a method for finding active substances for the treatment of pathological states based on lipid measurement parameter modulation/effector quotient profiles, in which the method according to the invention as defined above is carried out after the administration (i.e. the candidate active substance to be investigated is administered to the volunteer/organism before sample collection) or in the presence (i.e. the candidate active substance to be investigated is added to the sample after its withdrawal from the volunteer/organism) of a suitable candidate active substance. This candidate active substance may be regarded as being one of the “further modulating effectors” according to the invention. Of course, combinations of candidate active substances may also be used.

The invention further relates to a method for finding substances able to induce a pathological state as defined above based on lipid measurement parameter modulation/effector quotient profiles, in which the method according to the invention as defined above is carried out after the administration/application (i.e. the substance to be investigated is administered/applied to the volunteer/organism before sample collection) or in the presence (i.e. the substance to be investigated is added to the sample after its withdrawal from the volunteer/organism) of such a substance.

The invention further relates to a measuring instrument for carrying out the methods according to the invention as defined above, which has a surface on which the above-defined probes are immobilized.

Preferably, the probes form an addressable pattern on the surface, resulting in a so-called biochip.

In the following, the invention is illustrated in more detail without any limitation and by reference to precise examples.

In general, the invention relates to a method for the diagnosis or for the confirmation or exclusion of striking conditions, e.g. pathological states, or predispositions thereto, and to a method for monitoring the course of therapies and for finding active substances for the treatment of striking conditions, e.g. pathological states, which is based on the identification of a certain behaviour of tissues and cells with respect to the synthesis, release, reaction or the degradation of lipids such as eicosanoids, or to their receptors or degrading/synthesizing enzymes, that is both absolute and in mutual relation, wherein said behaviour is spontaneous, generally open for modulation (i.e. inducible or open for activation/inhibition) or specifically provoked.

In contrast to the state of the art, both the initial state (the native, non-modulated state corresponding to the zero value) as well as the modulated state (e.g. after stimulation with an indicator substance and at least one further modulator) of lipid measurement parameters, e.g. of the synthesized lipids/eicosanoids, the enzymes of the lipid/eicosanoid synthesis and/or the lipid/eicosanoid receptors, are investigated by the method according to the invention. Furthermore, at least two different lipid measurement parameters are determined, e.g. eicosanoids, eicosanoid enzymes and/or eicosanoid receptors of the same sample are analysed simultaneously. Hereby, not the static (initial/actual) state is taken, but in addition the dynamic/variability of the system to be investigated is characterized.

Such “balanced-score”-tests with respect to other parameters are already known in medicinal diagnostics for the evaluation of the immunological status, e.g. for the evaluation of the sub-populations of T-lymphocytes (Th1/Th2-ratios) in patients suffering from leprosy or HIV (41,42).

The following definitions for terms used herein are valid for the whole invention and in any combination.

Lipids according to the invention are for example saturated and in particular single as well as preferably polyunsaturated fatty acids of natural or synthetic origin (having at least 16 carbon atoms, e.g. 20 carbon atoms) as well as their natural and chemically/physically/technically induced derivatives and constructs.

Derivatives according to the invention (in particular with respect to the above lipids) are biological/natural, chemically induced, physically induced/synthesized descendants of the lipids. These may be formed from the lipids and/or the derivatives by enzymatic and/or non-enzymatic reactions (e.g. prostaglandin E1, prostaglandin E2, prostaglandin E3, leukotrienes, leukotriene C4-leukotriene D4-leukotriene D4; HETE (=Hydroxyeicosatetraenoic acid), PAF (“platelet activating factor”).

Constructs according to the invention (in particular with respect to the above lipids) are biologically and/or chemically manipulated lipids and/or derivatives with the addition or removal of chemical/physical/biological structures, i.e. constructs which are naturally not existing but created willingly by directed and/or undirected modification/synthesis in suitable systems (e.g. enzyme inhibitors, enzyme activators, in particular inhibitors and/or activators of the eicosanoid synthesis such as e.g. manipulators of the cyclooxygenases, lipoxygenases, receptor antagonists, receptor agonists, but also lipid constructs suitable for the detection, which can be detected and determined with fluorometers or luminometers or devices for measuring mass differences).

Organisms according to the invention are living and/or non-living multicellular and/or unicellular beings such as humans, animals, plants, fungi, bacteria and/or viruses and functional entities of these beings such as e.g. (but not exhaustive) organs, tissues, cell clusters, cells, cell components (e.g. mitochondria).

Samples according to the invention are manipulated or non-manipulated organisms and/or parts of/from one/several organisms with or without prior manipulation of the organism and/or released structures/substances (lipids), as well as derivatives and/or constructs of the modulated and/or non-modulated organism(s)/structure(s)/substance(s) (lipids) which are subjected to analysis with the aim of directly or indirectly establish at least two lipid measurement parameters. According to the invention, nucleic acids associated with the regulation/expression of lipids/eicosanoids, such as e.g. synthesizing and degrading enzymes as well as receptors, may be analysed as further samples.

Lipid measurement parameters according to the invention are units of a sample that can be qualified and/or quantified (e.g. lipid amounts, eicosanoid amounts, derivative amounts, construct amounts, enzyme amounts, receptor amounts, receptor densities, enzyme activities, nucleic acid amounts, receptor binding strengths, ligand stabilities in a sample or in a context to be defined).

A parameter according to the invention is a variable derived from one or more lipid measurement parameters for a direct or indirect evaluation or description of the status of the lipid ratio of the sample of the organism to be investigated, also enabling to predict the consequences of a further or envisaged modulation for the organism to be investigated.

Enzymes according to the invention are substances which are capable to modify a given substrate in its chemical/physical/biological nature and/or to intervene in chemical/physical/biological processes/reaction courses, hereby altering the substrates and, in particular, the lipids according to the invention (e.g. cyclooxygenases, lipoxygenases, monoxygenases).

Substrates according to the invention are substances that can be altered in their chemical/physical/biological propertie(s) by enzymes.

Receptors according to the invention are structures that reversibly and/or irreversibly bind lipids, derivatives, constructs, but also organisms or samples according to the invention. These can be naturally occurring structures (e.g. proteins) or else artificially generated structures/constructs without the necessity of having a biological function (except that they bind ligands). Receptors bind ligands and may have the same, equal, and/or similar chemical/physical/biological structure as ligands (i.e. prostaglandin E2 may be both a ligand as well as a receptor, depending on its behaviour in the biological context/in the organism to be investigated). For this, its chemical/physical/biological structure does not need to be known.

Ligands according to the invention are structures of natural and/or chemically/physically/biologically modified natural and/or artificially generated substances/constructs that are suitable to bind to receptors according to the invention and may have the same/equal/similar chemical/physical/biological structure as the receptors (e.g. proteins, peptides, saturated/unsaturated fatty acids and their derivatives and/or constructs, nucleic acids and/or nucleic acid derivatives/nucleic acid constructs). For this, its chemical/physical/biological structure does not need to be known.

A “modulator” or “effector” according to the invention is generally a matter or a substance or compound that can modulate, e.g. lower or elevate, one or more lipid measurement parameter(s) according to the invention. Thus, an effector can be both an inhibitor and activator, or an antagonist and agonist, respectively.

The method according to the invention generally comprises the following steps:
(a) withdrawal of a solid, fluid or gaseous sample (e.g. cells, tissue, fluid, breath) from an organism (e.g. human/animal/plant/bacterium);
(b) determination of the absolute and relative amounts of the lipids/eicosanoids and/or e.g. the regulators/effectors (e.g. enzymes/receptors) of these lipids/eicosanoids;
(c) determination of these lipids/eicosanoids/enzymes/receptors in the native (non-modulated) and/or modulated (stimulated/inhibited) state;
(d) differentiation of a risk constellation (or e.g. of the state of health/pathological state or of a confirmation or of an exclusion or diagnosis) on the basis of the ratio from (a) to (c) in groups of organisms/patients and/or individuals;
and, optionally,
(e) recording and evaluating the lipid/eicosanoid/enzyme/receptor status of the organism to be investigated and recording and evaluating the impact of therapeutic measures on the organism (therapy monitoring in vitro);
(f) recording and evaluating (risk constellation) the impact of the lipids/eicosanoids/enzymes/receptors on complex other systems to identify interactions (e.g. harmful substance-modulated bacteria or grass pollen and their effects on the samples to be analysed from the organism to be investigated).

Exemplifying flow chart for test implementation:
1. Sample withdrawal
2. Sample preparation
3. Sample exposition (modulating substance(s))
4. Measurement sample production
5. Measurement sample storage
6. Analytics (e.g. by EIA (“enzyme immunoassay”), RIA (“radio-ligand immunosorbent assay”), FIA (“fluorescence immuno-assay”), HPLC (“high pressure liquid chromatography”), GC (“gas chromatography”), 1H (“immunohistochemistry/immuno-cytochemistry”), WB (“western blotting”), NB (“northern blotting”), SB (“southern blotting”), GE (“gel electropho-resis”), PCR (“polymerase chain reaction”))
7. Collection of lipid measurement parameters (semi-quantitative/quantitative)
8. Measurement value processing
9. Establishment of the risk constellation (clinical interpretation, if desired).

The lipid/eicosanoid pattern or profile (different lipids/eicosanoids/enzymes/receptors) may be determined by using any samples (solid, fluid, gaseous) from any organism, wherein the measurement/analytical method applied is not subject to specific limitations and can be selected by the artisan in view of the practical situation.

Prior to analysis, the samples may have been modulated or non-modulated. The modulation may e.g. be effected by physical means (e.g. thermal radiation, nuclear radiation), chemical matters or substances (e.g. enzyme inhibitors/activators, receptor antagonists/agonists, specific or unspecific) or biological matters or substances (e.g. moulds, tree pollen, antibodies), which specifically or non-specifically influence the system to be investigated.

The samples may be/may be present in a biological matrix (body fluids such as serum, plasma, urine, stool, breath condensate or liquor; secretions of the glands of stomach, intestine, nose, lung, eye; cell homogenates; aerosols; eukaryotic and prokaryotic cells, tissue clusters and tissues) or in defined chemical solutions (e.g. cell culture medium, buffer/salt solutions, non-physiological solutions, e.g. in methanol).

The analytics may be performed quantitative or semi-quantitative, but it is always suitable for recording relative differences of the lipids/eicosanoids/enzymes/receptors/nucleic acids, either amongst manipulated samples for one of these parameters (e.g. non-stimulated prostaglandin E2 synthesis vs. stimulated and/or inhibited prostaglandin E2 synthesis) and/or for recording relative differences between certain metabolites (e.g. prostaglandin E2 vs. leukotriene D4, each non-modulated and modulated), to be defined in a particular case, to each other.

It is essential to always determine more than one lipid measurement parameter (e.g. before treatment/after treatment or non-modulated/modulated or eicosanoid 1/eicosanoid 2 or enzyme 1/enzyme 2, receptor 1/receptor 2, eicosanoid 1/receptor 1, eicosanoid 1/enzyme 1, receptor 1/enzyme 1, receptor 1/enzyme 2, etc.) from the same sample, which subsequently are at disposal for the further evaluation.

The lipids/eicosanoids are appropriately captured e.g. by enzyme immunoassay techniques, because this technology enables quickly and quantitative measurement of many samples.

Targets for measurement are e.g. lipids/eicosanoids (e.g. leukotrienes, prostaglandins, prostanoids, hydroxyeicosatetra-enoic acids, and other cell signaling lipids), lipid/eicosanoid receptors, enzymes of the lipid/eicosanoid synthesis or the corresponding degrading or regulating enzymes, and nucleic acids (mRNA) coding therefor.

In an apparatus embodiment of the invention, the functions may be accomplished by various means. The means for providing a sample may be an automated pipetting device or robot, a manually-operated pipette or syringe, a cell-sorting device, or the like automated dispensing device. The measuring means may include biochip technology as described below, optical readout (e.g. luminescence, fluorescence or absorbance measurement at a single wavelength or multiple wavelengths), radiological measurement, electrical measurement, and/or biochemical assays as well-known in the art and as described herein. The various measuring means may all be accomplished with a single element or a plurality of elements. The calculating means, means for obtaining standardized modulation quotient profile, means for obtaining standardized effector quotient profile, and comparing means may all be carried out by structures including a microprocessor connected to a storage device including electronic memory and/or magnetic media, preferably connected to a display device and/or output such as a printer. The various calculating means may all be accomplished with a single element or a plurality of elements. That is, the various means may be carried out by a single microprocessor with suitable software, or multiple microprocessors.

In addition to the already known, conventional methods (s.a.), the possibility to analyse lipids/eicosanoids by microarray or biochip technology is explicitly mentioned herein. Biochip technology enables the parallel semi-quantitative or quantitative determination of a plurality of the above-mentioned lipid measurement parameters, wherein the probes on the biochip for the determination of the lipid/eicosanoid measurement parameters may be grouped thematically (depending on the task to be solved) or else are grouped and applied according to the sought-after or given requirements (e.g. depending on the pathological state; e.g. pLT and PGE2 are important in one pathological state, whereas in another pLT and TXB2 or PGE2 and TXB2 or PGE2 und PGE2 receptors and/or cyclooxygenase-1 are important). The physical and/or chemical methods for the production of biochips by conventional techniques are not subject of any limitation.

The visualization of the lipids/eicosanoids/enzymes/receptors/nucleic acids to be detected or determined may particularly be effected by using fluorochromes or phosphorescent or bio/chemoluminescent or chromogenic substances.

The detection of analytical signals may e.g. be performed by means of optical and/or electrical measuring methods (e.g. potential modulation, conductivity modulation).

The object of the invention is the determination of lipid measurement parameter modulation/effector quotient profiles in order to characterize striking conditions (such as pathological states) and healthy conditions and thus constellations of risk factors, or e.g. to screen for anti-inflammatory matter or substances, e.g. phytopharmaceuticals, (“drug screening”), or to estimate the inflammatory potency in the assessment of the biocompatibility of implantable materials (product security/patient protection) or the predisposition for certain diseases (risk constellations) such as coagulation defects (e.g. thrombosis, pulmonary embolism), gastro-intestinal diseases (=intestinal diseases, e.g. ulcerative colitis, Crohn's disease, ulcer), food intolerances and inflammatory diseases of the brain (e.g. encephalitis). Further fields of application exist in birthing medicine, e.g. for clarifying, whether an abnormal course of birth, e.g. because of abnormal contractions, is to be expected.

The invention is illustrated in more detail in the following examples.

### EXAMPLE 1

By way of example, the determination of the basic state as well as of the stimulated states of prostaglandin E2 and peptide leukotrienes in analgesic intolerance is described.

Human leukocytes of the peripheral blood are separated from plasma with the aid of a dextran gradient, and adjusted to a defined cell count (100,000 cells/ml). Subsequently, the stimulation is effected by incubation of the cells, e.g. without or while adding arachidonic acid as modulating indicator substance, or anti-IgE as modulating effector for a defined time (30 minutes) at 37° C. in cell culture medium (e.g. RPMI 1640). After sedimentation of the cells by centrifugation for 5 minutes with 800×g at 4° C., the cell-free supernatant is collected and can be stored at −80° C. under nitrogen or argon, if desired. Afterwards, these measurement samples are transferred to a polystyrol plate coated with prostaglandin E2 or peptide leukotrienes, respectively, and incubated for 18 hours at 4° C. while adding an anti-prostaglandin-E2- or anti-peptide-leukotriene-antibody, respectively, (“competitive assay”). After a washing step, an incubation at 23° C. for 2 hours is performed with a biotinylated secondary antibody against the first antibody. After further washing, incubation follows with a streptavidin-conjugated peroxidase at 23° C. for 1 hour, and after further washing, a peroxidase substrate is added and the optical density is determined after about 30 minutes with a multichannel photometer. With the aid of the standard curves carried along, the measurement values can now be quantified and used for calculating the lipid measurement parameter stimulation/effector quotient profiles (an example for the calculation is given herein below).

An example for the pathological state of analgesic intolerance is the reduced basal synthesis of prostaglandin E2 (by 20% and more) in combination with an elevated basal peptide leukotriene synthesis (20% and more). The peptide leukotriene/prostaglandin E2 quotient is smaller 10. The synthesis of prostaglandin E2 induced by arachidonic acid is unstriking and the synthesis of peptide leukotriene induced by arachidonic acid is unstriking to faintly elevated (0-40%), if these values are compared with those from persons without pathological findings.

Only the determination of the lipid measurement parameter modulation/effector quotients enables differentiation among different eicosanoid patterns or profiles that can be attributed to different risk constellations (such as e.g. to patients having bronchial asthma or nasal and sinusal polyposis or analgesic induced asthma).

A suitable method for the determination of lipid measurement parameters is the fluorometric measurement of the degradation of unsaturated fatty acids/arachidonic acid which are stained with the dye 9-diethylamino-5H-[alpha]phenoxazin-5-one (38). The dye enters into living cells and is fluorescent as long as it can intracellularly bind to unsaturated fatty acids (i.e. those with 2 to more double bonds) (39-40). After activation of the cells, e.g. by LPS (lipopolysaccharide) or interleukin-1, activation of fatty acid degrading enzymes (e.g. phospholipase A2 (PLA)) occurs, causing a decline in fluorescence. In contrast, the fluorescence can also be elevated, if e.g. endogenic arachidonic acid is released by PLA from cell membranes into the cytoplasm and the degradation of the same does not take place.

Until now, 9-diethylamino-5H-[alpha]phenoxazin-5-one has only been used for histological/cytological investigations, but not for quantifying experiments.

In organisms with modified enzyme equipment, a modified degradation of the fatty acids (faster or slower) now occurs, which can be quantified by recording the fluorescence at different times (kinetics).

### EXAMPLE 2

100,000 leukocytes/ml are incubated in a 10⁻⁵ M solution of 9-diethylamino-5H-[alpha]phenoxazin-5-one in PBS solution for 15 minutes at room temperature. Afterwards, they are washed twice in PBS at 4° C. and then centrifuged with 600×g, before they are transferred to a reaction cuvette. A sample is now measured by fluorometry without further treatment (excitation wavelength at 485 nm, emission wavelength 570 nm). A further sample is e.g. stimulated with LPS (5 mg/ml) and measured by fluorometry. The fluorescence of both samples is recorded in time intervals of 1 minute up to a period of 60 minutes. From the values obtained, the increase and decline of fluorescence can be plotted graphically and the slopes of the curves can be determined by using suitable mathematical formulas.

In the case of the non-treated samples, a 0-5% increase in fluorescence occurs within the first 5-10 minutes, followed by a sigmoidal decline of fluorescence to 40-60% of the starting value after 60 minutes. In the case of the samples treated with LPS, there is a 5-20% increase in fluorescence within 5-10 minutes, and a 30-80% sigmoidal decline of fluorescence after 60 minutes.

On the basis of the values obtained, standardized lipid measurement parameter modulation/effector quotients may now be established or calculated and the profiles obtained may be compared with those from samples of reference organisms. If archive data from prior investigations already exist, these may be used.

Risk constellations for the investigated organism, e.g. with respect to its capability to metabolize unsaturated fatty acids, may then be established by comparison. From this, further therapeutic measures may then be derived.

This method can only be performed in vitro. Until now, the use of 9-diethylamino-5H-[alpha]phenoxazin-5-one for the quantification of the native and induced degradation of unsaturated fatty acids/arachidonic acid for the determination of constellations of risk factors has not been known.

A precise example for calculating standardized lipid measurement parameter modulation/effector quotients follows. Again, it is pointed out that the determination of measurement values for a standard group may be omitted, if corresponding archive data already exist.

### EXAMPLE 3

1) Leukocytes from the peripheral blood of investigation group 1/standard group/investigation group 2 are obtained by blood withdrawal, followed by density gradient separation (3% dextran solution) for separating plasma and leukocytes. The leukocytes thus obtained are washed 3 times with phosphate buffered solution (PBS) and pooled/resuspended in cell culture medium (RPMI 1650), before they are adjusted to a cell count of 100,000 cells/ml (investigation group 2 only in this example; in principle, a second group is not necessary for this test, but serves here for a better understanding and as second comparison group, both to the standard group and to the investigation group).

2) The cells are divided into 3 equal parts (e.g. 3 times with 1 ml cell suspension with 100,000 cells/ml each) in reaction vessels (e.g. plastic or glass vessels).

A control solution is added to the first part-sample (=without further modulator, =zero or blank value; control solution is the solvent, in which the modulators are dissolved).

Arachidonic acid is added to the second part-sample (concentration: 10⁻⁵ M; =indicator substance).

An anti-immunoglobulin-E solution is added to the third part-sample (e.g. 1:100 dilution of the anti-human-IgE-solution from DAKO, =modulator or modulating substance).

Exposition/incubation with the indicator substance arachidonic acid and the modulator anti-immunoglobulin-E-solution is started simultaneously at e.g. 37° C. for e.g. 30 minutes.

Thereafter, the cells are separated from the cell culture medium, e.g. by centrifugation at 4° C. with a sedimentation force of e.g. 800×g. The supernatant obtained from the 3 part-samples, separated for each part-sample, is then collected in suitable vessels (e.g. cryovessels) and stored until EIA analysis at e.g. −70° C.

3) The contents of prostaglandin E2 (PGE2) and peptide leukotrienes (pLT9) in the cell culture supernatants of the 3 part-samples are analysed and quantified by enzyme immunological assays (EIA) specific for PGE2 (=lipid measurement parameter A) and pLT (=lipid measurement parameter B), respectively.

From the first part-sample, values A₀ (e.g. 350±43 pg/ml PGE2) and B₀ (e.g. 120±9.7 pg/ml pLT) are obtained; from the second part-sample, values Aₘₐₓ (e.g. 4120±236 pg/ml PGE2) and Bₘₐₓ(e.g. 150±10.4 pg/ml pLT) are obtained; from the third part-sample, values A₂ (e.g. 1830±143.8 pg/ml PGE2) and B₂ (e.g. 83±5.7 pg/ml pLT) are obtained.

TABLE 1
Measurement values (in pg/ml)

| Measurement | Standard | Investigation | Investigation |
|---|---|---|---|
| parameter | group (N) | group 1 (I1) | group 2 (I2) |
| A₀ | 1820 ± 198 | 350 ± 43 | 2172 ± 207 |
| Aₘₐₓ | 4170 ± 275 | 4120 ± 236 | 3976 ± 245 |
| A₂ | 2975 ± 287 | 1830 ± 143 | 785 ± 69 |
| B₀ | 23 ± 3.5 | 120 ± 9.7 | 54 ± 5.8 |
| Bₘₐₓ | 143 ± 12.4 | 150 ± 10.4 | 128 ± 10.4 |
| B₂ | 43 ± 5.7 | 83 ± 5.7 | 135 ± 12.7 |

4) Calculation of the modulation quotients for the lipid parameter A (=PGE2) and the lipid parameter B (=pLT) is performed on the basis of the values obtained from the part-samples:

TABLE 2
Modulation quotients

| Measurement | Standard | Investigation | Investigation |
|---|---|---|---|
| parameter | group(n) | group 1 (i1) | group 2 (i2) |
| Aₘₐₓ/A₀ | 2.3 | 11.8 | 1.8 |
| A₂/A₀ | 1.6 | 5.2 | 0.4 |
| Bₘₐₓ/B₀ | 6.2 | 1.3 | 2.4 |
| B₂/B₀ | 1.9 | 0.7 | 2.5 |

(values of table 2 which are striking when compared to the control are marked in bold)

The modulation quotients allow distinguishing the investigation groups (i1, i2) from the standard group (n), but not to differentiate the investigation groups (i1, i2).

5) Standardization by division by the corresponding modulation quotients of the standard group (n).

TABLE 3
Standardized modulation quotients

| Measurement | Standard | Investigation | Investigation |
|---|---|---|---|
| Parameter | group(n) | group 1 (i1) | group 2 (i2) |
| Aₘₐₓ/A₀ | 1 (0.8-1.2) | 5.1 | 0.78 |
| A₂/A₀ | 1 (0.5-1.5) | 3.3 | 0.25 |
| Bₘₐₓ/B₀ | 1 (0.5-1.2) | 0.21 | 0.38 |
| B₂/B₀ | 1 (0.5-1.2) | 0.37 | 1.32 |

(in table 3, striking values are marked in bold)

The standardized modulation quotients also allow only to distinguish the investigation groups (i1, i2) from the standard group (n), but not to differentiate the investigation groups (i1, i2). Here, the differentiation of the investigation groups depends on the standard group, i.e. also two or more patient groups which are otherwise similar, may be differentiated, e.g. if a “healthy” standard group can not be established, or if it is to be clarified whether the two investigation groups differ from each other.

6) Effector quotients (calculation as above)

TABLE 4
Effector quotients (PGE/pLT)

| Measurement | Standard | Investigation | Investigation |
|---|---|---|---|
| parameter | group(n) | group 1 (i1) | group 2 (i2) |
| A₀/B₀ | 80.4 | 2.9 | 40.2 |
| Aₘₐₓ/Bₘₐₓ | 29.2 | 27.5 | 31.1 |
| A₂/B₂ | 69.2 | 22.1 | 5.8 |

(values of table 4 which are striking when compared to the control are marked in bold)

With the aid of the second modulator (=anti-IgE), the effector quotients allow to differentiate the two investigation groups (i1, i2). Furthermore, the differences between both investigation groups and the standard group become more clear.

7) Standardized effector quotients (calculation as above)

TABLE 5
Standardized effector quotients

| Measurement | Standard | Investigation | Investigation |
|---|---|---|---|
| Parameter | group(n) | group 1 (i1) | group 2 (i2) |
| A₀/B₀ | 1 (0.5-1.2) | 0.036 | 0.50 |
| Aₘₐₓ/Bₘₐₓ | 1 (0.9-1.1) | 0.941 | 1.065 |
| A₂/B₂ | 1 (0.2-1.2) | 0.319 | 0.084 |

(values of table 4 which are striking when compared to the control are marked in bold)

The standardized effector quotients allow to clearly differentiate the two investigation groups (i1, i2) vs. the standard group (n), as well as to differentiate the two investigation groups against each other. Here, the values given partly depend on the standard group selected, but another standard group (n-x) could also be selected. This can e.g. be done in order to differentiate several investigation groups (i1, i2, i3, i4) against each other. If no “standard group” is available or can be established, an investigation group (n4) may be selected as “standard group”. This alternative may also be applied, if e.g. differentiation against “treated” (t1) and “otherwise treated” (t2) and/or “non-treated” (t3=n-x) group is desired.

8) Remarks: If necessary, the measurement values of the pLT determinations are to be corrected with a “compensation factor” (may be determined experimentally and ranges between 5 and 100), whereby the values derived/determined therefrom would change on a relational basis.

9) Conclusions from the results obtained:

Investigation group 2 is classified in risk constellation 2, i.e. there is an analgesic intolerance without an allergic component. This means that a desactivation against non-steroidal analgesics is recommended, and that people of this group should avoid non-steroidal antiphlogistics until desactivation is successfully completed. Investigation group 1 is classified in risk constellation 1, i.e. there is an analgesic intolerance with a profound allergic-related component. This means that an allergometry should be performed on people of this group, followed by desensitization, if necessary. Only after this, desactivation against analgesics could be appropriate.

While the present invention has been described with reference to certain illustrative embodiments, one of ordinary skill in the art will recognize that additions, deletions, substitutions and improvements can be made while remaining within the scope and spirit of the invention as defined by the appended claims.

Literature

1) Hirschberg V. G. S.
Mitteilung über einen Fall von Nebenwirkungen des Aspirin. Dt Med Wochenschr 1902;28:416.
2) von Euler U.S.
Über die spezifische blutdrucksenkende Substanz des menschlichen Prostata- und Samenblasensekretes.
Klin Wschr 1935;14:1182-1186.
3) Hanahan D. J.
Platelet activating factor: A biological active phosphoglyceride.
Ann Rev Biochem 1986;55:483-509.
4) Hamberg M., Svensson J., Samuelsson B.
Thromboxanes: a new group of biologically active compounds derived from prostaglandin endoperoxidase.
Proc Natl Acad Sci USA 1975;72:2994-2998.
5) Vane J. R.
Inhibition of prostaglandin biosynthesis as a mechanism of action for aspirin-like drugs.
Nature (New Biol) 1971;231:232-235.
6) Bergström S.
Prostaglandines: Members of a new hormonal system.
Science 1967;157:382-390.
7) Samuelsson B.
Some recent advances in leukotriene research.
Adv Exp Med Biol 1997;433:1-7.
8) Samuelsson B., Dahlen S.-E., Lindgren J. A. Rouzer C. A, Serhan C. N.
Leukotrienes and lipoxins: structures, biosynthesis, and biological effects.
Science 1987;237:1171-1176.
9) Holzman J. Y.
Arachidonic acid metabolism.
Am Rev Respir Dis 1991; 143:188-203.
10) Smith W. L.
The eicosanoids and their biochemicaal mechanisms of action.
Biochem J 1989; 259;315-324.
11) Fradin A., Zirrolli J. A., Maclouf J., Vausbinder L., HENSON P. M., Murphy R. C.
Platelet-activating factor and leukotriene biosynthesis in whole blod—a model for the study of transcellular arachidonic metabolism.
J Immunol 1989;143;3680-3685.
12) Smith D. L & Willis A. L.
A suggested shorthand nomenclature for the eicosanoids.
Lipids 1987;22:983-987.
13) Corey E. J., Niwa H., Falck J. R., Mioskowski C., Arai Y., Marfat A.
Recent studies on the chemical synthesis of eicosanoids.
Adv Prostaglandin Thromboxane Res 1980;6:19-25.
14) Willis A. L. & Smith D. L.
Metabolism of arachidonic acid.
in: The handbook of immunopharmacology. Lipid mediators, ed.: Cunningham F. M., Academic Press, London, 1994:1-32.
15) Slater T. F. & McDonald-Gibson R. G.
Introduction to the eicosanoids.
in: Prostaglandins and related substances, eds.: Benedetto C, McDonald-Gibson RG, Nigam S, Slater TF, IRL Press, Oxford, 1987:1-4.
16) Rowley A. F., Kuhn H., Schewe T.
Eicosanoids and related compounds in plants and animals.
PoPrinceton University Press, Princeton, 1998.
17) Stanley-Samuelson D. W.
Physiological role of prostaglandins and other eicosanoids in invertebrates.
Biol Bull 1987;173:92-109.
18) Bell M. V, Henderson R. J., Sargent J. R.
The role of polyunsaturated fatty acid in fish.
Comp Biochem Physiol 1986;85B:711-719.
19) Vick B. A.
Oxygenated fatty acids of the lipoxygenase pathway.
Chapter 5
in: Lipid metabolism in plants.
ed: Moore T. S.
CRC Press, Boca Raton 1993:167-191.
20) Dennis E. A.
Diversity of group types, regulation, and function of phospholipase A2.
J Biol Chem 1994;269:13057-13063.
21) Creminon C., Habib A., Maclouf J., Pradelles P., Grassi J.
Differential measurement of constitutive (COX-1) and inducible (COX-2) cyclooxygenase expression in human umbilical cells using specific immunometric enzyme immunoassays.
Biochim Biophys Acta 1995;1254:341-348.
22) O'Neil G. P. & Ford-Hutshinson A. W.
Expression of mRNA for cyclooxygenase-1 and cyclooxygenase 2 in human tissue.
FEBS Lett 1993;330:156-160.
23) Kennedy I., Coleman R. A., Humphrey P. P. A., Levy H. P., Lumley P.
Studies on the characterisation of prostanoid receptors: a proposed classification Prostaglandins, 1982: 24:667-689.
24) DeWitt D. L & Meade E. A.
Serum and glucocorticoid regulation of gene transcription and expresssion of prostaglandin H synthase-1 and prostaglandin H synthase-2 isoenzymes.
Arch Biochem Biophys 1993;306:94-102.
25) Gardiner P. J. Abram T. S. Tudhope S. R. Cuthbest N. J. Norman P., Brink C.
Leukotriene receptors and their selective antagonists.
Adv Prstaglandin Thromboxane Leukot Res 1994;2:49-61.
26) Hong J. L. & Lee L.-Y.
Cigaret smoke-induced bronchoconstriction: causative agents and role of thromboxane receptors.
J Appl Physiol 1995;78:2260-2266.
27) Coleman R. A., Smith W. L., Narumiya s.
Classification of the prostanoid receptors: properties, distribution, and structure of the receptors and their subtypes.
Pharmacol rev. 1994;46:205-229.
28) Taylor G. W. & Wellings R.
Measurements of fatty acids and their metabolites.
in: Lipid mediators
ed.: Cunningham F. M.
Accademic Press, London 1994:33-60.
29) Kulmacz R. J., & Lands W. E. M.
Cyclooxygenase: Measurement, purification and properties.
in: Prostaglandins and related substances.
eds.: Benedetto C., McDonald-Gibson R. G., Nigam S., Slater T. F.
Oxford University Press, Oxford 1989:209-228.
30) Schewe T., Kuhn H., Rapoport S. M.
Lipoxygenases: measurement, charaterization, and properties.
in: Prostaglandins and related substances.
eds.: Benedetto C., McDonald-Gibson R. G., Nigam S., Slater T. F.
Oxford University Press, Oxford 1989:229-242.
31) Vilaseca J., Salas A., Guarner F., Rodriguez R., Malagelada J. R.
Participation of thromboxane and other eicosanoid synthesis in the course of experimental inflammatory colitis.
Gastroenterology 1990;98:269-277.
32) De Weck A. L., Stadler B. M., Urwyler A., Wehner H. U., Bühlmann R. P.
Cellular antigen stimulation test (CAST)-A new dimension in allergy diagnostics.
Allergy Clin Immunol News 1993;5:9-14.
33) Baenkler H.-W., Schäfer D., Hosemann W.
Eicosanoids from biopsies of normal and polyposis nasal mucosa.
Rhinology 1996;34:166-170.
34) Schäfer D., Lindenthal U., Wagner M., Bölcskei P. L., Baenkler H. W.
Effect of prostaglandin E2 on eicosanoid release by human bronchial biopsy specimens from normal and inflamed mucosa.
Thorax 1996;51:919-932.
35) Sauer S. K., Schäfer D., Kress M., Reeh P. W.
Stimulated prostaglandin E2 from rat skin, in vitro. Life Science 1998;62:2045-2055.
36) Schäfer D., Schmid M., Göde U., Baenkler H.-W.
Dynamics of eicosanoids in peripheral blood cells during bronchial provocation in aspirin-intolerant asthmatics.
Eur Respir J 1999; 13:638-646.
37) Westcott J. Y.
The measurement of leukotrienes in human fluids.
Clin Rev Allergy Immunol 1999;17:153-177.
38) Greenspan P., Mayer E. P., Fowler S. D.
Nile Red: a selective fluorescent stain for intracellular lipid droplets.
J. Cell Biol. 1985;100:965-973.
39) Dvorack A. M., Dvorak H. F., Peters S. P., Shulman E. S., Mac Glashan D. W., Pyne K., Harvey V. S., Galli S. J., Lichtenstein L. M.
Lipid bodies: cytoplasmatic organelles important to arachidonate metabolism in macrophages and mast cells.
J Immunol 1983;131:2965-2976.
40) Weller P. F. & Dvorack A. M.
Arachidonic acid incorporation by cytoplasmatic lipid bodies of human eosinophils.
Blood 1985;65:1269-1274.
41) Erdmann E.
Klinische Kardiologie.
Springer Verlag, Berlin, 2000.
42) Adler G., Beglinger C., Müller-Lissner S., Schmigel W.
Klinische Gastroenterologie und Stoffwechsel.
Springer Verlag, Berlin, 2000.

1. A method for the diagnosis of pathological states or predispositions thereto based on lipid measurement parameter modulation/effector quotient profiles, comprising the steps of:
(a) providing a sample from an organism to be investigated and dividing the sample into a plurality of sufficient equal part-samples to allow for measurement of a plurality of values for each of a plurality of lipid measurement parameters A, B, C, . . . ;
(b) measuring a plurality of zero values A₀, B₀, C₀, . . . in the absence of a modulating effector; measuring a plurality of indicator values Aₘₐₓ, Bₘₐₓ, Cₘₐₓ, . . . in the presence of a modulating effector or an indicator substance; and measuring a plurality of values for a further modulation A₂, B2, C₂, . . . in the presence of a further modulating effector;
(c) calculating a plurality of quotients of the measurements Aₘₐₓ/A₀, A₂/A₀; BₘₐₓB₀, B₂/B₀; Cₘₐₓ/C₀, C₂/C₀; . . . for each lipid measurement parameter A, B, C, . . . of the sample from the organism to be investigated; and dividing the quotients by the corresponding values of one or more standard group(s), resulting in standardized modulation quotients which in their totality form a standardized modulation quotient profile for the organism to be investigated;
(d) calculating a plurality of quotients A₀/B₀, B₀/A₀, A₀/C₀, C₀/A₀, B₀/C₀, C₀/B₀ . . . in any combination from the zero values A₀, B₀, C₀ . . . ; and a plurality of quotients Aₘₐₓ/Bₘₐₓ, Bₘₐₓ/Aₘₐₓ, Aₘₐₓ/Cₘₐₓ, Cₘₐₓ/Aₘₐₓ, Bₘₐₓ/Cₘₐₓ, Cₘₐₓ/Bₘₐₓ . . . in any combination from the indicator values Aₘₐₓ, Bₘₐₓ, Cₘₐₓ . . . ; and a plurality of quotients A₂/B₂, B₂/A₂, A₂/C₂, C₂/A₂, B₂/C₂, C₂/B₂ . . . in any combination from the values for further modulation A₂, B₂, C₂ . . . ; and then dividing the values obtained for the organism to be investigated by a plurality of corresponding values obtained for one or more standard group(s) to obtain a plurality of standardized effector quotients which in their totality form a standardized effector quotient profile for the organism to be investigated; and
(e) diagnosing, confirming, or excluding a constellation of risk factors, a pathological state, or a predisposition thereto by comparing the standardized modulation quotient profile and the standardized effector quotient profile of the organism to be investigated with that of a corresponding investigation group in which the constellation of risk factors of interest, the pathological state, or the predisposition is present.

2. Method according to claim 1, wherein in step (a) said lipid measurement parameters are selected from the group consisting of measurement parameters for unsaturated fatty acids, degrading enzymes and synthesizing enzymes for unsaturated fatty acids, nucleic acids coding for degrading enzymes and synthesizing enzymes for unsaturated fatty acids, receptors for unsaturated fatty acids, and nucleic acids coding for receptors for unsaturated fatty acids.

3. Method according to claim 2, wherein said unsaturated fatty acids are selected from the group consisting of platelet-activating factor and eicosanoids.

4. Method according to claim 3, wherein said eicosanoids are selected from the group consisting of peptide leukotrienes, prostaglandin E2, thromboxane A2 and thromboxane B2.

5. Method according to claim 1, where said modulating effector or indicating substance of step (b) is selected from the group consisting of arachidonic acid, chemotactic peptides, anti-IgE, lipopolysaccharide, and interleukin.

6. Method according to claim 1, wherein said further modulating effector used in step (a) is a substance which may cause a pathological state or is involved in the onset or development thereof.

7. Method according to claim 6, wherein said pathological state is selected from tumours, cystic fibrosis, polyposis, bronchial asthma, an intolerance, coagulation defects, overcoming of infection, and an inflammation.

8. Method according to claim 6, wherein said pathological state is inflammatory and neoplastic change of the gastrointestinal tract.

9. Method according to claim 6, wherein said intolerance is a food, food additive or drug intolerance or an allergy, or wherein said coagulation defects represent the basis for thromboses or haemorrhages or thrombophilia, or wherein said overcoming of infection is a resistance to bacterial or viral or mycotic elements, e.g. associated with bacterial, viral or mycotic mucositis, or wherein the inflammation is encephalitis, sinusitis, rhinitis, neurodermatitis, Crohn's disease or ulcerative colitis.

10. Method according to claim 9, wherein said drug intolerance is an analgesic intolerance or said allergy is a pollen, spore, mite, wasp or bee venom allergy.

11. Method according to claim 10, wherein said analgesic intolerance is intolerance of acetylsalicylic acid.

12. Method according to claim 1, where one or more, optionally labeled eicosanoid(s) or the dye 9-diethylamino-5H-[alpha]phenoxazin-5-one is/are used to determine the lipid measurement parameters.

13. Method according to claim 1, where immobilized probes are used to determine the lipid measurement parameters, and the immobilized probes are selected from the group consisting of antibodies or functional fragments thereof against degrading enzymes or synthesizing enzymes of unsaturated fatty acids or against receptors for unsaturated fatty acids, and nucleic acids which hybridize onto nucleic acids which code for degrading enzymes or synthesizing enzymes of unsaturated fatty acids or for receptors for unsaturated fatty acids.

14. Method according to claim 13, wherein said antibodies are selected from the group consisting of polyclonal, monoclonal and single-chain antibodies, and said nucleic acids are selected from cDNA, mRNA and oligonucleotides.

15. Method according to claim 13, where the immobilized probes form an addressable pattern on a surface.

16. Method for monitoring the course of therapies of pathological states based on lipid measurement parameter modulation/effector quotient profiles, in which a method according to claim 1 is carried out after the administration or in the presence of a suitable medicament.

17. Method for finding active substances for the treatment of pathological states based on lipid measurement parameter modulation or effector quotient profiles, in which a method according to claim 1 is carried out after the administration or in the presence of a candidate active substance.

18. Method for finding substances able to induce a pathological state based on lipid measurement parameter modulation or effector quotient profiles, in which a method according to claims 1 is carried out after an administration/application or in the presence of such a substance.

19. The method of claim 1, wherein said sample contains leukocytes.

20. The method of claim 1, wherein said lipid measurement parameters are selected from the group consisting of measurement parameters for ceramide; ceramide-1-phosphate; sphingosine; sphingosine-1-phosphate; phosphatidic acid; diacylglycerol; lysophosphatidic acid; the phosphatidylinositol phosphates; and enzymes modifying ceramide, ceramide-1-phosphate, sphingosine, sphingosine-1-phosphate, phosphatidic acid, diacylglycerol, lysophosphatidic acid, or the phosphatidylinositol phosphates.

21. An apparatus for obtaining lipid measurement parameter modulation or effector quotient profiles, comprising:
(e) means for providing a sample from an organism;
(f) means for measuring a plurality of values for each of a plurality of lipid measurement parameters A, B, C, . . . , the means for measuring comprising:
i. means for measuring a plurality of zero values A₀, B₀, C₀, . . . in the absence of a modulating effector;
ii. means for measuring a plurality of indicator values Aₘₐₓ, Bₘₐₓ, Cₘₐₓ, . . . , in the presence of a modulating effector or an indicator substance; and
iii. means for measuring a plurality of values for a further modulation A₂, B2, C₂, . . . , in the presence of a further modulating effector;
(g) means for calculating, the calculating means comprising:
i. means for obtaining a standardized modulation quotient profile, comprising a plurality of standardized modulation quotients, by dividing plurality of quotients of the measurements Aₘₐₓ/A₀, A₂/A₀; BₘₐₓB₀, B₂/B₀; Cₘₐₓ/C₀, C₂/C₀; . . . for each lipid measurement parameter A, B, C, . . . of the sample from the organism to be investigated and dividing the quotients by the corresponding values of one or more standard group(s); and
ii. means for obtaining a standardized effector quotient profile, comprising a plurality of standardized effector quotients, by calculating a plurality of quotients A₀/B₀, B₀/A₀, A₀/C₀, C₀/A₀, B₀/C₀, C₀/B₀ . . . in any combination from the zero values A₀, B₀, C₀ . . . ; and a plurality of quotients Aₘₐₓ/Bₘₐₓ, Bₘₐₓ/Aₘₐₓ, Aₘₐₓ/Cₘₐₓ, Cₘₐₓ/Aₘₐₓ, Bₘₐₓ/Cₘₐₓ, Cₘₐₓ/Bₘₐₓ . . . in any combination from the indicator values Aₘₐₓ, Bₘₐₓ, Cₘₐₓ . . . , and a plurality of quotients A₂/B₂, B₂/A₂, A₂/C₂, C₂/A₂, B₂/C₂, C₂/B₂ . . . in any combination from the values for further modulation A₂, B2, C₂ . . . ; and then dividing the values obtained for the organism to be investigated by a plurality of corresponding values obtained for one or more standard group(s); and
(h) means for comparing the standardized modulation quotient profile and the standardized effector quotient profile of the organism to be investigated with that of a corresponding investigation group.

22. The apparatus of claim 21, wherein one or more of said measuring means comprises a surface on which probes defined for determination of the lipid measurement parameters are immobilized, which probes are selected from the group consisting of antibodies or functional fragments thereof against degrading enzymes or synthesizing enzymes of unsaturated fatty acids, antibodies or functional fragments thereof against receptors for unsaturated fatty acids, and nucleic acids which hybridize onto nucleic acids which code for degrading enzymes or synthesizing enzymes of unsaturated fatty acids or for receptors for unsaturated fatty acids, wherein the antibodies are preferably selected from polyclonal, monoclonal and single-chain antibodies, and the nucleic acids are preferably selected from cDNA, mRNA and oligonucleotides.

23. The apparatus of claim 22, wherein the probes form an addressable pattern on the surface.
